(19) United States
(12) Patent Application Publication
(10) Pub. No.: US 2005/0009120 A1
(43) Pub. Date: Jan. 13, 2005

(54) Methods of detecting ovarian cancer based on osteopontin

(75) Inventors: Samuel C. Mok, Brookline, MA (US); Bin Ye, Brookline, MA (US); Daniel W. Cramer, Chestnut Hill, MA (US)

Correspondence Address: FITCH, EVEN, TABIN & FLANNERY, P. O. BOX 65973, WASHINGTON, DC, 20035, US

(73) Assignee: The Brigham and Women's Hospital, Inc.

(21) Appl. No.: 10/869,027

(22) Filed: Jun. 17, 2004

Related U.S. Application Data
(63) Continuation-in-part of application No. 09/948,094, filed on Sep. 7, 2001.
(60) Provisional application No. 60/231,166, filed on Sep. 7, 2000.

(51) Int. Cl. G01N 33/574; C07H 21/04

(52) U.S. Cl. 435/7.23

(57) ABSTRACT

The present invention is directed to diagnostic methods based upon the expression of the protein osteopontin. In particular, it is concerned with assays of urine samples collected from women for the purpose of determining whether they are at increased risk for having ovarian cancer.

### CROSS REFERENCE TO RELATED APPLICATIONS

The present application is a continuation-in-part of U.S. application Ser. No. 09/948,094, filed on Sep. 7, 2001. application Ser. No. 09/948,094 claims the benefit of U.S. provisional application No. 60/231,166, filed on Sep. 7, 2000 (now abandoned).

### STATEMENT OF GOVERNMENT FUNDING

The United States Government has a paid-up license in this invention and the right in limited circumstances to require the patent owner to license others in reasonable terms as provided for by the terms of NIH Grant No. U01CA86381-01 awarded by the Department of Health and Human Services.

### FIELD OF THE INVENTION

The present invention is in the field of tumor cell markers and is particularly concerned with methods of detecting ovarian cancer by assaying urine samples for osteopontine. Any method for determining osteopontin levels may be used, including assays of the protein, N-, and C-terminal fragments of the protein, or modified forms of the protein.

### BACKGROUND OF THE INVENTION

Ovarian cancer is the fifth leading cause of death from cancer in U.S. women. In most instances, a diagnosis is not made until the cancer is in an advanced state; at a time when the five year survival rate of patients is only about 28% (Ries, et al., SEERC Cancer Stat. Rev. 1973-1995 (1998)). In contrast, the five year survival rate for women diagnosed with localized disease is about 95%. These statistics provide an incentive to search for tests that can be used to identify ovarian cancer at an early stage.

Osteopontin is a secreted phosphoprotein produced by a variety of cells and is found in normal plasma, urine, milk and bile (U.S. Pat. No. 6,414,219; U.S. Pat. No. 5,695,761; Denhart, et al., FASEB J 7:1475-1482 (1993); Oldberg, et al., Proc. Natl. Acad. Sci. USA 83:8819 (1986); Oldberg, et al., J. Biol. Chem. 263:19433-19436 (1986); Giachelli, et al., Trends Cardiovasc. Med. 5:88-95 (1995)). The protein has been known by a variety of different names (see U.S. Pat. No. 6,686,444 and U.S. Pat. No. 5,695,761), including OPN (Wranca, et al., Nucl. Ac. Res. 17:3306 (1989); 2ar (Smith, et al., J. Cell Biochem. 34:10-22 (1987); transformation-associated secreted phosphoprotein (Singer, et al., Anticancer Res. 48:1291 (1989); and Early T-lymphocyte activator-1 (Patarca, et al., Proc. Natl. Acad. Sci. USA 88:2736 (1991)). The human osteopontin protein and cDNA have been isolated and sequenced (Kiefer, et al., Nucl. Ac. Res. 17:3306 (1989). Elevated levels of osteopontin have been reported to be present in the sera of patients with advanced metastatic disease (U.S. Pat. No. 6,686,444), and to be expressed by carcinomas and sarcomas (Singer, et al., Anticancer Res. 48:1291 (1989)).

### SUMMARY OF THE INVENTION

The present invention is based upon the discovery that assays of the concentration of osteopontin in urine samples can be used for identifying patients who have, or are likely to develop ovarian cancer. Human osteopontin has been completely sequenced and both polyclonal and monoclonal antibodies that recognize this protein are commercially available (e.g., from R&D Systems Inc., Minneapolis, Minn.; and Abcam Ltd., Cambridge, Mass.). Methods for detecting this protein have been described in the art and ELISA assays for quantitating the human protein are commercially available (Assay Designs Inc., Ann Arbor, Mich.). It is possible that the osteopontin in urine may be partially degraded or in a modified form due to processing by the body prior to excretion. For the purposes herein, it will be understood that assays for determining osteopontin levels which detect fragments of osteopontin or modified forms of osteopontin are included within the scope of the invention.

In its first aspect, the invention is directed to a method of diagnostically evaluating a woman for the presence of ovarian cancer. This is accomplished by obtaining a urine sample from the woman and then assaying it for the concentration of osteopontin present. Any method for quantitating osteopontin is compatible with the method including immunoassays, surface enhanced laser desoprtion/ionization-mass spectrometry; chromatography; and electrophoretic methods. The most preferred method employs an enzyme-linked immunosorbent assay (ELISA). Results obtained from the assay of the urine sample are compared with similar results obtained from assays of one or more control samples and it is concluded that the woman from which the urine sample was obtained is at increased risk of having ovarian cancer if the concentration of osteopontin in her sample is higher than the amount found in said control samples. Methods for selecting appropriate controls are well known in the art. For example, controls may be urine samples obtained from women believed to be free from malignant disease or they may simply be urine samples obtained from the general population of women.

The optimum difference in osteopontin concentration between an obtained and control sample for the purpose of identifying women at increased risk for ovarian cancer will be determined using methods well known in the art. In general, this involves balancing the desire to identify a high percentage of women having ovarian cancer with the desire to avoid an excessive number of false positives. For example, an osteopontin concentration higher than the mean of normal control samples by at least one or two standard deviations may be used as a criteria for separating women that are “at risk” from women that are not. Similarly women having at least 2-5 times the normal concentration of osteopontin in their urine may be identified as being at increased risk, with individuals having levels over 5 times normal being especially at risk. However, adjustments may be made in these values as the experience is obtained using different types of tests for osteopontin.

The diagnostic method described above may also be combined with other diagnostic methods for ovarian cancer to improve reliability. The additional test may involve an imaging procedure or involve assaying for a different tumor cell marker. The most preferred of the tumor cell marker assays is for CA 125 in the serum or urine of a patient.

### DETAILED DESCRIPTION OF THE INVENTION

The discovery of a correlation between the concentration of osteopontin in urine and ovarian cancer is, in part, an outgrowth of more extensive studies on genes that are either over- or underexpressed in cancerous ovarian epithelial cells. Numerous genes were identified which may also potentially be used as markers and which are listed in the tables found in the Examples section below. Further testing on urine samples obtained from patients having ovarian cancer, benign ovarian disease and from healthy patients led to the conclusion that osteopontin, either alone or in combination with other markers, provides a good method for screening patients for ovarian cancer. It is believed that tests of other biological fluids (e.g., serum or plasma, or ovarian fluid) will also show a correlation between osteopontin concentration and risk for ovarian cancer and that the assays described herein may be used as a prognostic indicator and for ascertaining the effect of treatment methods as well as for indicating the presence of ovarian cancer.

The complete amino acid and nucleotide sequence of human osteopontin is known (see Kiefer, et al., Nucl. Ac. Res. 17:3306 (1989)) and assays for quantitating the concentration of osteopontin in a biological sample are commercially available (Assay Designs Inc., Ann Arbor, Mich.). Any type of quantitative assay is compatible with the invention including immunoassay procedures performed using, for example, a commercially available ELISA assay or an immunoassay developed using commercially available monoclonal or polyclonal antibodies. Alternatively, the osteopontin protein may be purified and immunoassays may be developed based upon the development of new antibodies that bind specifically to this protein.

Antibodies that bind specifically to osteopontin are defined for the purpose of the present invention as those that have at least a 100 fold greater affinity for osteopontin than for any other similar undenatured protein. The process for producing such antibodies may involve either injecting the osteopontin protein itself into an appropriate animal or injecting short peptides made to correspond to different regions of osteopontin. The peptides injected should be a minimum of 5 amino acids in length and should be selected from regions believed to be unique to the protein. Methods for making and detecting antibodies are well known to those of skill in the art as evidenced by standard reference works such as: Harlow, et al., Antibodies. A Laboratory Manual, Cold Spring Harbor Laboratory, NY (1988); Klein, Immunology: The Science of Self-Nonself Discrimination (1982); Kennett, et al., Monoclonal Antibodies and Hybridomas: A New Dimension in Biological Analyses (1980); and Campbell, “Monoclonal Antibody Technology,” in: Laboratory Techniques in Biochemistry and Molecular Biology (1984).

“Antibody” as used herein is meant to include intact molecules as well as fragments which retain the ability to bind antigen (e.g., Fab and F(ab′) fragments). These fragments are typically produced by proteolytically cleaving intact antibodies using enzymes such as a papain (to produce Fab fragments) or pepsin (to produce F(ab′)₂ fragments). The term “antibody” also refers to both monoclonal antibodies and polyclonal antibodies. Polyclonal antibodies are derived from the sera of animals immunized with the antigen. Monoclonal antibodies can be prepared using hybridoma technology (Kohler, et al., Nature 256:495 (1975)). In general, this technology involves immunizing an animal, usually a mouse, with either intact osteopontin or a fragment derived from osteopontin. The splenocytes of the immunized animals are extracted and fused with suitable myeloma cells, e.g., SP₂O cells. After fusion, the resulting hybridoma cells are selectively maintained in HAT medium and then cloned by limiting dilution (Wands, et al., Gastroenterology 80:225-232 (1981)). The cells obtained through such selection are then assayed to identify clones which secrete antibodies capable of binding to osteopontin.

The antibodies or fragments of antibodies described above may be used to detect to the presence of the osteopontin in any of a variety of immunoassays. For example, antibodies may be used in radioimmunoassays or immunometric assays, also known as “two-site” or “sandwich assays” (see Chard, “Introduction to Radioimmune Assay and Related Techniques,” in: Laboratory Techniques in Biochemistry and Molecular Biology, North Holland Publishing Co., N.Y. (1978)). In a typical immunometric assay, a quantity of unlabelled antibody is bound to a solid support that is insoluble in the fluid being tested. After the initial binding of antigen to immobilized antibody, a quantity of detectably labeled second antibody (which may or may not be the same as the first) is added to permit the detection and/or quantitation of bound antigen (see e.g., Radioimmune Assay Method, Kirkham, et al., ed., pp. 199-206, E&S Livingston, Edinburgh (1970)). Many variations of these types of assays are known in the art and may be employed for the detection of osteopontin.

If desired, antibodies to osteopontin may also be used in the purification of the protein (see generally, Dean, et al., Affinity Chromatography, A Practical Approach, IRL Press (1986)). Typically, antibody is immobilized on a chromatographic matrix such as Sepharose 4B. The matrix is then packed into a column and the preparation containing osteopontin is passed through under conditions that promote binding, e.g., under conditions of low salt. The column is then washed and bound osteopontin is eluted using a buffer that promotes dissociation of antibody, e.g., a buffer having an altered pH or salt concentration. The eluted osteopontin may be transferred into a buffer of choice, e.g., by dialysis, and either stored or used directly.

### EXAMPLES

### Example 1

### Differentially Exposed Genes from Ovarian Cancer Cells

A. Materials and Methods

Cell Culture

Cultures of normal human ovarian surface epithelial cells (HOSE) were established by scraping the HOSE cells from the ovary and growing them in a mixture of Medium 199 and MCDB105 supplemented with 10% fetal calf serum (Mok, et al., Gynecol. Oncol. 52:247-52, (1994)). The seven HOSE cells used were HOSE17, HOSE636, HOSE642, HOSE695, HOSE697, HOSE713, and HOSE726. Ovarian cancer cell lines used were OVCA3, OVCA420, OVCA432, OVCA433, OVCA633, SKOV3, and ALST.

Microarray Probe and Hybridization

MICROMAX™ human cDNA microarray system I (NEN Life Science Products, Inc., Boston, Mass.), which contains 2400 known human cDNAs on a 1×3″ slide, was used in this study. Microarray probe and hybridization were performed as described in the instruction manual. In brief, biotin-labeled cDNA was generated from 3 μg total RNA, which was pooled from HOSE17, HOSE636 and HOSE642. Dinitrophenyl (DNP)-labeled cDNA was generated from 3 μg total RNA that was pooled from ovarian cancer cell lines OVCA420, OVCA433 and SKOV3. Before the cDNA reaction, an equal amount of RNA control was added to each batch of the RNA samples for normalization during data analysis. Biotin-labeled and DNP-labeled cDNA were mixed, dried and resuspended in 20 μl hybridization buffer, which was added to the cDNA microarray and covered with a coverslip. Hybridization was carried out overnight at 65° C. inside a hybridization cassette.

Post Hybridization and Cyanine-3 (Cy3™) and Cyanine-5 (Cy5™) Tyramide Signal Amplification (TSA)

After hybridization, microarrays were washed with 30 ml 0.5×SSC, 0.01% SDS, and then 30 ml 0.06×SSC, 0.01% SDS. Finally the microarray was washed with 0.06×SSC. Hybridization signal from biotin-labeled cDNA was amplified with streptavidin-horseradish peroxidase and Cy5™-tyramide, while hybridization signal from DNP-labeled cDNA was amplified with anti-DNP-horseradish peroxidase and Cy3™-tyramide. After signal amplification and post-hybridization wash, cDNA microarray was air-dried and detected with a laser scanner.

Image Acquisition and Data Analysis

Cy3 signal was derived from ovarian cancer cells and Cy5 signal was derived from HOSE cells. Laser detection of the Cy3 and Cy5 signal on the microarray was acquired with a confocal laser reader, ScanArray3000 (GSI Lumonics, Watertown, Mass.). Separate scans were taken for each fluor at a pixel size of 10μ. cDNA derived from the control RNA hybridized to 12 specific spots within the microarray. Cy3 and Cy5 signals from these 12 spots should theoretically be equal and were used to normalize the different efficiencies in labeling and detection with the two fluors. The fluorescence signal intensities and the Cy3/Cy5 ratios for each of the 2400 cDNAs were analyzed by the software Imagene 3.0 (Biodiscovery Inc, Los Angeles, Calif.).

Real-Time Quantitative RT-PCR

Real-time PCR was performed in duplicate using primer sets specific to GA733-2, osteopontin, prostasin, creatine kinase B, CEA, KOC and a housekeeping gene, cyclosporin, in an ABI PRISM 5700 Sequence Detector. RNA was first extracted form normal ovarian epithelial cell cultures (HOSE695, 697, 713, and 726) and six ovarian carcinoma cell lines (OVCA3, OVCA432, OVCA433, OVCA633, SKOV3 and ALST). cDNA were generated from 1 μg total RNA using the TaqMan reverse transcription reagents containing 1× TaqMan RT buffer, 5.5 mM MgCl₂, 500 μM dNTP, 2.5 μM random hexamer, 0.4 U/μl RNase inhibitor, 1.25 U/μl MultiScribe reverse transcriptase (PE Applied Biosystems, Foster City, Calif.) in 100 μl. The reaction was incubated at 25° C. for 10 min, 48° C. for 30 min and finally at 95° C. for 5 min. 0.5%1 of cDNA was used in a 20 μl PCR mix containing 1×SYBR PCR buffer, 3 mM MgCl₂, 0.8 mM dNTP, and 0.025 U/μl AmpliTaq Gold (PE Applied Biosystems, Foster City, Calif.). Amplification was then performed with denaturation for 10 min at 95° C., followed by 40 PCR cycles of denaturation at 95° C. for 15 sec and annealing/extension at 60° C. for 1 min. The changes in fluorescence of SYBR Green I dye in every cycle was monitored by the ABI5700 system software, and the threshold cycle (C_{T}) for each reaction was calculated. The relative amount of PCR products generated from each primer set was determined based on the threshold cycle or C_{T} value. Cyclosporin was used for the normalization of quantity of RNA used. Its C_{T} value was then subtracted from that of each target gene to obtain a ΔC_{T} value. The difference (ΔΔC_{T}) between the ΔC_{T} values of the samples for each gene target and the ΔC_{T} value of the calibrator (HOSE726) was determined. The relative quantitative value was expressed as 2^{-ΔΔCT}.

B. Results and Discussion

The MICROMAX system

The MICROMAX system allows the simultaneous analysis of the expression level of 2400 known genes. The use of TSA signal amplification in the system after hybridization reduces the amount of total RNA needed to a few micrograms which is about 20-100 times less than currently used methods. The details of TSA have been described previously for chromosome mapping of PCR-labeled probes less than 1 kb by FISH (Schriml, et al., Biotechniques 27:608-611, (1999)). In this study, 30 putative differentially over-expressed genes (excluding 9 ribosomal genes) were identified in ovarian cancer cell lines (Table 1). Using high density cDNA array on membranes, Schummer et al. (Schummer, et al., Gene 238:375-385, (1999)) has identified 32 known genes that exhibit a tumor-to-HOSE ratios of more than 2.5-fold. Fourteen of these 32 genes were present in the MICROMAX cDNA microarray but only five of them were present at more than 3-fold.

Biotin-labeled cDNA was made from ovarian cancer cell lines, while DNP-labeled cDNA was made from HOSE cells. The differential TSA amplification of the hybridization signal depends on the use of a Steptavidin-HRP conjugate or anti-DNP-HRP conjugate in a sequential step. At each step, cyanine-5-Tyramide or cyanine-3-Tyramide can be added and the HRP will then catalyze the deposit of Cy3 or Cy5 onto the hybridized cDNA nonspecifically. As a result, either Cy3 or Cy5 signals can be used for the cDNA derived from ovarian cancer cell lines, and vice versa for HOSE cells. Thus, it is not necessary to make two different sets of probes to compare the effect of Cy3 or Cy5 fluorescence as a result of their differences in extinction coefficients and quantum yields. Cy3 and Cy5 signals on the processed slides were stable for more than 6 months.

Normalization of Signals

The MICROMAX system has 3 nonhuman genes as internal controls. Each of the control genes is spotted 4 times on the microarray. Equal amounts of polyA RNA derived from these control genes were spiked into the total RNA samples derived from both HOSE and ovarian cancer cell lines during cDNA synthesis. Thus, hybridization signals from these control genes in two RNA samples should theoretically be the same. The Cy3 to Cy5 ratios for these control genes varied from 0.4 to 4.0 and the average ratio was 1.5±1.1. From a prior microarray analysis of human cancer cells, 88 genes have been identified to express at relatively constant levels in many cell types (DeRisi, et al., Nat. Genet. 14:457-60, (1996)). The MICROMAX microarray also contains 58 of these 88 genes and 21 of these genes with signal to background ratio more than 3-fold were analyzed (Table 2). The ratios varied from 0.23 to 5.22. The average ratio is 1.6±1.5. Thus, the result of internal control RNA for normalizing signal was similar to that of genes that express at a relatively constant level in different cell types.

Effect of Background Signal on the Identification of Differentially Expressed Genes

In the present study, 1357 of the 2400 genes on the microarray have Cy3 signals (from ovarian cancer cell lines) that were at least two-fold higher than the background, and 740 genes have Cy3 signals that were at least three-fold higher than the background. After post-hybridization washes, there was still significant background intensity for the Cy3 signal but very low background for Cy5. Subsequently, the microarray was washed again in 30 ml TNT buffer at 42° C. for 20 minutes instead of at room temperature, followed by 30 ml of 0.006×SSC for 1 minute. The washed microarray was then dried and re-scanned. This process was repeated several times until the number of genes with signal to background ratios at least 3-fold remained the same. The extensive washing steps decreased the background intensity significantly, but there was no obvious changes in the signal intensity. As a result, the number of genes with at least 3-fold signal to background ratios increased from 740 to 791 genes. Moreover, the differential expression ratios, in general, also increased (Table 1 to Table 2). More importantly, after the extensive washing, we were able to detect the differential expression of two weakly expressed genes, thiol-specific antioxidant protein (4.5-fold) and elongation factor-1-β (9.7-fold), which were previously identified by Schummer et al. Thus, the extensive post-hybridization washing and re-scanning of signals may be necessary to decrease background signal especially in the case of differentially expressed genes with low expression levels.

Confirmation of Differential Expression by Real-Time Quantitative PCR

To further validate differential expression, five interesting genes were chosen, GA733-2, osteopontin, koc, prostasin, and creatine kinase B, for real-time PCR analysis. All these genes are either surface antigens or secreted proteins. Thus, they may be useful as tumor markers for ovarian cancer. GA733-2 is a cell surface 40-kDa glycoprotein associated with human carcinomas of various origins (Szala, et al., Proc. Natl. Acad. Sci. USA 87:3542-6, (1990)). Osteopontin is a secreted glycoprotein with a conserved Arg-Gly-Asp (RGD) integrin-binding motif and is expressed predominantly in bone, but has also been found in breast cancer and thyroid carcinoma with enhanced invasiveness (Sharp, et al., Lab. Investigat. 79:869-877, (1999), Tuck, et al., Oncogene 18:4237-4246, (1999)). Prostasin is a novel secreted serine proteinase which was originally identified in seminal fluid (Yu, et al., J. Biol. Chem. 269:18843-8, (1994)). The koc transcript is highly over-expressed in pancreatic cancer cell lines as well as in pancreatic cancer. It is speculated that koc may assume a role in the regulation of tumor cell proliferation by interfering with transcriptional and or posttranscriptional processes (Mueller-Pillasch, et al., Oncogene 14:2729-33, (1997)). Creatine kinase B is a serum marker associated with renal carcinoma and lung cancer (Kurtz, et al., Cancer 56:562-6, (1985), Takashi, et al., Urologia Internationalis 48:144-8, (1992)). Two randomly selected genes, CEA and RGS, were used as negative controls.

The results showed that all the tested ovarian cancer cell lines expressed higher levels of GA733-2. However, osteopontin, prostasin, KOC and creatine kinase B were over-expressed in only some of the cancer cell lines. Since we were using pools of RNA, the differential expression that was observed is an average of the gene expression from 3 independent HOSE cells or 3 different cancer cell lines. This strategy allows us to capture genes that overexpress in either some or all of the cell lines. Genes that only overexpress in some of the ovarian cancer cell lines may be useful for molecular classification of ovarian cancer cells. Since as little as 10 pg cDNA is enough for real-time quantitative RT-PCR reaction, RNA extracted from microdissected tissue would be enough for thousands of such real-time quantitative RT PCR analyses.

TABLE 1
List of genes differentially over-expressed in ovarian
cancer cells more than 10-fold.

| | | Before | After | |
|---|---|---|---|---|
| | | extensive | extensive | |
| | | washing | washing | Cy3 signal |
| Accession # | Description | (Cy3/Cy5) | (Cy3/Cy5) | intensity |
| M33011 | carcinoma-associated antigen | 472 | 444 | 1249 |
| | GA733-2 | | | |
| J04765 | Osteopontin | 156 | 184 | 11851 |
| L41351 | Prostasin | 44 | 170 | 3172 |
| L19783 | GPI-H | 4 | 88 | 916 |
| U96759 | Von Hippel-Lindau binding protein | 60 | 59 | 1377 |
| | (VBP-1) | | | |
| M57730 | B61 | 20 | 49 | 5514 |
| L33930 | CD24 signal transducer and 3′ | 24 | 47 | 26722 |
| | region | | | |
| D55672 | hnRNP D | 45 | 44 | 950 |
| U97188 | Putative RNA binding protein KOC | 223 | 38 | 3599 |
| L19871 | ATF3 | 9 | 37 | 3507 |
| J04991 | p18 | 15 | 34 | 9914 |
| D00762 | mRNA for proteasome subunit HC8 | 17 | 29 | 4703 |
| U17989 | Nuclear autoantigen GS2NA | 5 | 28 | 721 |
| U43148 | Patched homolog (PTC) | 10 | 28 | 4155 |
| AF010312 | Pig7(PIG7) | 13 | 23 | 17379 |
| M80244 | E16 | 18 | 21 | 4180 |
| X99802 | mRNA for ZYG homologue | 14 | 21 | 2086 |
| U05598 | Dihydrodioldehydrogenase | 10 | 18 | 21595 |
| L47647 | Creatine kinase B. | 7 | 18 | 787 |
| M55284 | Protein kinase C-L (PRKCL) | 7 | 16 | 863 |
| X15722 | mRNA for glutathione reductase | 23 | 14 | 794 |
| 554005 | Thymosin beta-10 | 6 | 13 | 1476 |
| AB006965 | mRNA for Dnmlp/Vpslp-like | 7 | 13 | 4183 |
| | protein | | | |
| M83653 | Cytoplasmic phosphotyrosyl protein | 6 | 13 | 2156 |
| | Phosphatase | | | |
| X12597 | mRNA for high mobility group-1 | 7 | 12 | 2785 |
| | protein | | | |
| | (HMG-1) | | | |
| M18112 | poly(ADP-ribose) polymerase | 6 | 12 | 9277 |
| U56816 | Kinase Mytl (Mytl) | 4 | 11 | 1773 |
| X06233 | mRNA for calcium-binding protein | 7 | 11 | 3007 |
| | In macrophages (MRP-14) | | | |
| D85181 | mRNA for fungal sterol-C5- | 6 | 11 | 3571 |
| | desaturase | | | |
| | homolog | | | |
| M31627 | X box binding protein-1 (XBP-1) | 5 | 10 | 12151 |

TABLE 2
Cy3 versus Cy5 ratio for a set of genes that are previously
shown to express at relative constant level (2)

| | | Before extensive | After extensive |
|---|---|---|---|
| | | washing | washing |
| Accession # | Description | (Cy3/Cy5) | (Cy3/Cy5) |
| X06323 | MRL3 mRNA for ribosomal protein L3 | 3.31 | 5.22 |
| | homologue | | |
| AF006043 | 3-phosphoglycerate dehydrogenase | 3.81 | 4.8 |
| M37400 | Cytosolic aspartate aminotransferase | 3.03 | 3.66 |
| D30655 | mRNA for eukaryotic initiation factor | 4.17 | 3.48 |
| | 4A11 | | |
| J04208 | inosine-5′-monophosphate dehydrogenase | 1.13 | 2.15 |
| | (IMP) | | |
| M17885 | Acidic ribosomal phosphoprotein PO | 2.74 | 2.09 |
| X54326 | mRNA for glutaminyl-tRNA synthetase | 1.17 | 2.01 |
| J04973 | Cytochrome bc-1 complex core protein II | 0.98 | 1.6 |
| D13900 | mitochondrial short-chain enoyl-CoA | 0.91 | 1.52 |
| | hydratase | | |
| Z1531 | mRNA for elongation factor 1-gamma | 0.76 | 0.89 |
| D78361 | mRNA for ornithine decarboxylase | 0.51 | 0.82 |
| | antizyme | | |
| U13261 | e1F-2 associated p67 homolog | 0.41 | 0.82 |
| X15183 | mRNA for 90-kDa heat-shock protein | 0.8 | 0.75 |
| M36340 | ADP-ribosylation factor 1 (ARF1) | 0.5 | 0.66 |
| X91257 | mRNA for seryl-tRNA synthetase | 0.75 | 0.52 |
| AF047470 | Malate dehydrogenase precursor (MDH) | 0.41 | 0.51 |
| | mRNA | | |
| D13748 | mRNA for eukaryotic initiation factor | 0.33 | 0.43 |
| | 4A1 | | |
| L36151 | Phosphatidylinositol4-kinase | 0.26 | 0.38 |
| X04297 | mRNA for Na, K-ATPase alpha-subunit | 0.27 | 0.34 |
| X79535 | mRNA for beta tubulin, clone nuk_278 | 0.18 | 0.28 |
| J04173 | Phosphogylcerate mutase (PGAM-B) | 0.14 | 0.23 |

TABLE 3
Real-time quantitative RT-PCR analysis of a few selected genes^{a}.

| | GA733- | | | | Creatin | | |
|---|---|---|---|---|---|---|---|
| | 2 | Osteopontin | KOC | Prostasin | Kinase B | CEA | RGS |
| Normal ovarian | | | | | | | |
| cells | | | | | | | |
| HOSE695 | 4 | 21 | 5 | 28 | 0.4 | 5 | 32 |
| HOSE697 | 1 | 1 | 2 | 4 | 0.4 | 3 | 18 |
| HOSE713 | 1 | 20 | 7 | 5 | 1 | 16 | 25 |
| HOSE726 | 1 | 1 | 1 | 1 | 1 | 1 | 1 |
| Average (HOSE) | 2 | 11 | 4 | 9 | 1 | 6 | 19 |
| Ovarian cancer | | | | | | | |
| cell lines | | | | | | | |
| OVCA3 | 419 | 6 | 4 | 61 | 393 | 1 | 4 |
| OVCA432 | 136 | 0 | 0 | 17 | 8 | 0 | 1 |
| OVAC433 | 2048 | 0 | 52 | 57 | 12 | 1 | 4 |
| OVCA633 | 2917 | 13777 | 3 | 228 | 4 | 15 | 27 |
| SKOV3 | 2856 | 265 | 10 | 2 | 31 | 6 | 13 |
| ALST | 3875 | 6081 | 78 | 10 | 1 | 2 | 5 |
| Average | 2042 | 3355 | 24 | 62 | 75 | 4 | 9 |
| (OVCA) | | | | | | | |
| OVCA/HOSE | 1361 | 310 | 6 | 7 | 103 | 1 | 0.5 |
| (average) | | | | | | | |

^{a}Each gene was analyzed using an identical panel of 10 cDNA samples that comprised of 4 normal ovarian surface epithelial cells and 6 ovarian cancer cell lines. The expression of each gene for each cDNA sample was normalized against cyclosporin. Duplicated reactions were performed for each of the genes and similar results were obtained.

### Example 2

### Differentially Expressed Genes

A. Choice of Samples and the Identification of Differentially Expressed Genes

We compared the expression of 2400 genes between primary human ovarian surface epithelial (HOSE) cells and ovarian cancer (OVCA) cells using the MICROMAX™ cDNA microarray system (NEN Life Science Products, Boston, Mass., USA). Three primary HOSE cells from different individuals were pooled together as a normal sample. The use of pooled normal samples has two advantages—(1) fluctuations in gene expression among normal HOSE cells due to the individual difference in age or physiological states may be minimized, and (2) a sufficient amount of RNA for direct labeling can be obtained from the precious primary cell cultures. Similarly, three different cancer cell lines were pooled together as an ovarian cancer sample for the analysis.

47 genes over-expressed (Table 4 and Table 5), and 58 genes down-regulated in ovarian cancer cells (Table 6) were identified from a single microarray experiment. However, the list of genes described here is different from two similar studies reported previously (Schummer, et al., Gene 238:375 (1999), Wang, et al., Gene 229:101, (1999)). Only a few differentially expressed genes were shared by these studies. The differences in the list of differentially expressed genes may be due to the use of different samples in the analysis. We compared the gene expression of primary normal ovarian surface epithelial cells and ovarian cancer cell lines. In one of the previous studies, gene expression of normal ovary was compared with tumor tissues, while gene expression of low passage ovarian surface epithelial cells were compared with tumor tissues in another study. Apparently, the choice of samples for analysis would account for the different set of genes identified.

B. Background Hybridization Signal and the Identification of Weakly Expressed Genes

Gene expression from OVCA samples was detected as a Cy3 signal while gene expression from HOSE samples was detected as Cy5 signal. After completion of the recommended procedures, significant background signal was still observed in the Cy3 signal that derived from OVCA sample. A series of additional, stringent post-hybridization washes reduced the background signal. While the Cy3 to Cy5 ratios for most of the genes increased slightly after stringent post-hybridization washes, the ratios, for some genes increased significantly. More importantly, after the stringent post-hybridization washes, we were able to detect the weakly expressed genes that are differentially over-expressed in ovarian cancer cells (Table 5).

C. Genes Over-Expressed in Ovarian Cancer Cells

From the list of over-expressed genes, several putative mechanisms may be involved in the pathogenesis of ovarian cancer—(1) inactivation of a tumor suppressor, (2) altered expression of transcription factors, (3) overexpression of oncogenes, (4) overexpression of glycosylphosphatidylinositol (GPI) anchor associated proteins, and (5) altered cell cycle control. According to this list of genes, VBP1 interacts with the product of the von Hippel-Lindau gene and is expected to participate in pathways by inactivation of this tumor suppressor gene. RNA binding proteins, Koc and hnRNP D, may assume a role in the regulation of tumor cell proliferation by interfering with transcriptional and/or posttranscriptional processes of tumor suppressor genes. However, the precise role of these RNA binding proteins in human tumor cells remains to be elucidated. ATF3 and XBP-1 are transcription factors which may play an important role in the regulation of gene expression by cAMP-dependent intracellular signaling pathways and be essential for hepatocyte growth respectively. Also related to gene transcription, HMG-I protein has been implicated as a potential marker for thyroid carcinoma. p18 and E16 are two oncogenes that have been found to be over-expressed in acute leukemia cells and various human cancers respectively. The glycosylphosphatidylinositol (GPI) anchor, potentially capable of generating a number of second messengers, such as diacylglycerol, phosphatidic acid, and inositol phosphate glycan, has been postulated to be involved in signal transduction in various cell types, including T-cells. Genes encoding GPI anchored proteins. (GPI-H, B61 and CD24) were found to be over-expressed in ovarian cancer cells. Mytl activity is temporally regulated during the cell cycle and is suggested to play a role in mitotic control. CD24, a GPI anchored protein, is also involved in cell cycle control.

The differential expression of five interesting genes, GA733-2, osteopontin, koc, prostasin, and creatine kinase B, has previously been confirmed by real-time RT-PCR. All these genes are either surface antigens or secreted proteins, which may be potential serum markers. In fact, we have found that prostasin is significantly higher in the plasma of an ovarian cancer patient. GA733-2 is known as epithelial cell surface antigen (EPG) or adenocarcinoma-associated antigen (KSA). These proteins may function as growth factor receptors. Osteopontin is an acidic phosphorylated glycoprotein of about 40 Kd which is abundant in the mineral matrix of bones and possibly functions as a cell attachment factor involved in tumor invasion and metastasis. Prostasin is a serine proteinase expressed in prostate and prostate carcinoma. Creatine kinase has been shown to be at an elevated level in the blood of patients with renal cell carcinoma or small lung carcinoma.

D. Genes Down-Regulated in Ovarian Cancer Cells

More than 50 genes down-regulated in ovarian cancer cells were identified in this study. In this list of genes (Table 6), SPARC/osteonectin has been previously identified as a down-regulated gene. SPARC is an extracellular matrix (ECM) protein with tumor-suppressing activity in human ovarian epithelial cells (Mok, et al., Oncogene 12:1895, (1996)). Other ECM or ECM related proteins such as fibronectin, tenascin, OB-cadherin-1, HXB, matrix metalloproteinase, and ICAM-1 were also found to be down-regulated. Tenascin has been suggested to be a prognostic marker for colon cancer. Patients with more tenascin expression have better long-term survival than patients with no or weak expression.

Several other genes involved in responding to growth factors or mitogens were also down-regulated. These genes were Shps-1, phosphorylase-kinase, phosphoinositide 3-kinase, NDP kinase, ZIP-kinase, signal transducing guanine nucleotide-binding regulatory protein, IGFBP2, TGF-beta, and TNFα receptor. SHPS-1, a novel glycoprotein, binds the Sh2-domain-containing protein tyrosine phosphatase SHP-2 in response to mitogens and cell adhesion. Suppression of SHPS-1 expression by v-Src via the Ras-MAP kinase pathway has been shown to promote the oncogenic growth of cells. NDP kinase gene located on chromosome 17q has been proposed as a metastasis suppressor gene in a variety of tumor types. ZIP kinase is a novel serine/threonine kinase and has been shown to mediate apoptosis through its catalytic activities. Previous work suggests that the TGF-beta receptor complex and its downstream signaling intermediates constitute a tumor suppressor pathway. The stabilization of TNF-alpha receptors on the surface of human colon carcinoma cells is necessary for TNFα induced cell death. Besides these two major groups of genes, other genes encoding proteases and complement Cl components were also down-regulated. Some of these down-regulated genes, such as testican and osteoblast specific factor 2, have not yet been associated with carcinogenesis.

TABLE 4
Overexpressed Genes

| | | | OVCA |
|---|---|---|---|
| Accession | | (OVCA/ | signal |
| # | Description | HOSE) | intensity |
| M33011 | carcinoma-associated antigen | 444 | 1249 |
| | GA733-2 | | |
| 704765 | Osteopontin | 184 | 11851 |
| L41351 | Prostasin | 170 | 3172 |
| L19783 | GPI-H | 88 | 916 |
| U96759 | Von Hippel-Lindau binding protein | 59 | 1377 |
| | (VBP-1) | | |
| M57730 | B61 | 49 | 5514 |
| L33930 | CD24 signal transducer and 3′ region | 47 | 26722 |
| D55672 | hnRNP D | 44 | 950 |
| U97188 | Putative RNA binding protein KOC | 38 | 3599 |
| L19871 | ATF3 | 37 | 3507 |
| 704991 | p18 | 34 | 9914 |
| D00762 | proteasome subunit HC8 | 29 | 4703 |
| U17989 | Nuclear autoantigen GS2NA | 28 | 721 |
| U43148 | Patched homolog (PTC) | 28 | 4155 |
| AF010312 | Pig7 (PIG7) | 23 | 17379 |
| M80244 | E16 | 21 | 4180 |
| X99802 | ZYG homologue | 21 | 2086 |
| U05598 | Dihydrodiol dehydrogenase | 18 | 21595 |
| L47647 | Creatine kinase B. | 18 | 787 |
| M55284 | Protein kinase C-L (PRKCL) | 16 | 863 |
| X15722 | glutathione reductase | 14 | 794 |
| 554005 | Thymosin beta-10 | 13 | 1476 |
| AB006965 | Dnmlp/Vpslp-like protein | 13 | 4183 |
| M83653 | Cytoplasmic phosphotyrosyl protein | 13 | 2156 |
| | phosphatase | | |
| X12597 | high mobility group-1 protein | 12 | 2785 |
| | (HMG-1) | | |
| M18112 | poly(ADP-ribose) polymerise | 12 | 9277 |
| U56816 | KinaseMytl (Mytl) | 11 | 1773 |
| X06233 | calcium-binding protein in | 11 | 3007 |
| | macrophages (MRP-14) | | |
| D85181 | fungal sterol-C5-desaturase homolog | 11 | 3571 |
| M31627 | X box binding protein-1 (XBP-1) | 10 | 12151 |

TABLE 5
Weakly expressed genes identified after stringent washes.

| | | | |
|---|---|---|---|
| AF005654 | Actin-binding double-zinc-finger protein | 18770 | 751 |
| | (abLIM). | | |
| L10844 | Cellular growth-regulating protein. | 33 | 725 |
| M88163 | Global transcription activator homologous | 18 | 642 |
| | sequence. | | |
| U02882 | Rolipram-sensitive 3′,5′-cyclic AMP | 27 | 636 |
| | phosphodiesterase. | | |
| X12517 | U1 small nuclear RNP-specific C protein. | 112 | 550 |
| D29833 | Salivary proline rich peptide P-B. | 10 | 492 |
| AF020918 | Glutathione transferase GSTA4 | 47 | 475 |
| J05262. | Farnesyl pyrophosphate synthetase | 95 | 469 |
| L08424 | Achaete scute homologous protein | 57 | 457 |
| | (ASHI). | | |
| M84526. | Adipsin/complement factor D | 65 | 441 |
| U35113 | Metastasis-associated mtal. | 13 | 367 |
| D28468 | DNA-binding protein TAXREB302. | 268 | 357 |
| AF012126 | Zinc finger protein (ZNF198). | 15 | 342 |
| ABOO0714 | RVP1. | 11 | 314 |
| AF029750 | Tapasin (NGS-17). | 118 | 305 |
| X60489 | Elongation factor-1-beta. | 10 | 282 |
| L36645 | Receptor protein-tyrosine kinase (HEK8). | 71. | 273 |

TABLE 6
List of genes down-regulated in ovarian cancer cells more
than 10-fold.

| | | (HOSE/ | HOSE |
|---|---|---|---|
| Accession # | Description | OVCA) | signal |
| D45421 | phosphodiesterase I alpha | R | 667 |
| M35410 | Insulin-like growth factor binding | R | 1517 |
| | protein 2 (IGFBP2) | | |
| X81334 | collagenase-3 protein | R | 5146 |
| D13665 | osteoblast specific factor 2 (OSF-2pl) | R | 24300 |
| J03040 | SPARC/osteonectin | R | 28711 |
| D86043 | SHPS-1 | 681 | 9450 |
| U89942 | Lysyl oxidase-related protein (WS9-14) | 454 | 25055 |
| M59807 | NK4 | 118 | 22438 |
| Z74616 | Prepro-alpha2(I) collagen | 101 | 2281 |
| Z74615 | Prepro-alpha1(I) collagen | 81 | 24323 |
| X06596 | Complement component Cls | 76 | 22672 |
| M95787 | 22 kDa smooth muscle protein (SM22) | 71 | 31581 |
| X06256 | Fibronectin receptor alpha subunit | 66 | 27901 |
| M36981 | Putative NDP kinase (nm23 H2S) | 60 | 15411 |
| AJ001838 | Maleylacetoacetate isomerase | 59 | 304 |
| X56160 | Tenascin | 42 | 36062 |
| D21254 | OB-cadherin-1 | 40 | 20621 |
| Y07921 | Serine protease | 37 | 2247 |
| Y10032 | Putative serine/threonine protein kinase | 36 | 8510 |
| X04526 | Beta-subunit signal transducing | 36 | 13321 |
| | proteins Gs/Gi (beta-G) | | |
| L31409 | Creatine transporter | 35 | 8677 |
| X04701 | Complement component Clr | 33 | 10299 |
| X13839 | Vascular smooth muscle alpha-actin | 32 | 27311 |
| X84908 | Phosphorylase-kinase, beta subunit. | 30 | 11341 |
| L14595 | Alanine/serine/cysteine/threonine | 27 | 2234 |
| | transporter (ASCTI) | | |
| M97796 | Helix-loop-helix protein (Id-2) | 25 | 3187 |
| X04741 | Protein gene product (PGP) 9.5 | 25 | 13138 |
| Y10055 | Phosphoinositide 3-kinase | 23 | 614 |
| X83535 | Membrane-type matrix | 20 | 6464 |
| | metalloproteinase | | |
| U69546 | RNA binding protein Etr-3 | 18 | 2714 |
| U16268 | AMP deaminase isoform L, | 18 | 3512 |
| | alternatively spliced (AMPD2) mRNA, | | |
| | exons 1B, 2 and 3. | | |
| S59749 | 5E10 antigen | 18 | 1249 |
| U03057 | Actin bundling protein (HSN) | 17 | 9285 |
| J02854 | 20-kDa myosin light chain (MLC-2) | 16 | 8323 |
| X16940 | Enteric smooth muscle gamma-actin | 16 | 18776 |
| X13223 | N-acetylglucosamide-(beta 1-4)- | 16 | 3357 |
| | galactosyltransferase | | |
| M69181 | Nonmuscle myosin heavy chain-B | 16 | 11126 |
| | (MYH10) | | |
| X06990 | ICAM-1 | 16 | 25605 |
| M13656 | Plasma protease (C1) inhibitor | 15 | 1091 |
| X73608 | Testican | 15 | 2977 |
| M96803 | General beta-spectrin (SPTBNI) | 15 | 13359 |
| D00632 | Glutathione peroxidase | 15 | 4951 |
| X03445 | Nuclear envelope protein lamin C | 13 | 17451 |
| | precursor | | |
| L06419 | Lysyl hydroxylase (PLOD) | 13 | 9288 |
| M12125 | Fibroblast muscle-type tropomyosin | 12 | 34379 |
| S45630 | Alpha B-crystallin, Rosenthal fiber | 12 | 1536 |
| | component. | | |
| AB005298 | BAI 2 | 11 | 3839 |
| L77864 | Stat-like protein (Fe65) | 11 | 1914 |
| AB007144 | ZIP-kinase | 11 | 4277 |
| M16538 | Signal-transducing guanine nucleotide- | 11 | 2793 |
| | binding regulatory (G) protein beta | | |
| | subunit | | |
| L35545 | Endothelial cell protein C/APC | 11 | 1002 |
| | receptor (EPCR) | | |
| M75161 | Granulin | 11 | 13289 |
| X69910 | p63 | 11 | 16460 |
| D12686 | eIF-4 gamma | 11 | 21555 |
| L07594 | Transforming growth factor-beta type | 11 | 672 |
| | III receptor (TGF-beta) | | |
| M33294 | Tumor necrosis factor receptor | 10 | 6592 |
| U18121 | 136-kDa double-stranded RNA binding | 10 | 1619 |
| | protein p136 (K88dsRBP) | | |
| M55618 | Hexabrachion (HXB) | 10 | 3866 |

### Example 3

### Prostasin as a Serum Marker for Ovarian Cancer

A. Materials and Methods

Biological Specimens

Ovarian tissue and cells were freshly collected from women undergoing surgery at the Brigham and Women's Hospital for diagnosis of primary ovarian cancer or from control subjects having a hysterectomy and ophorectomy for benign disease. Cultures of normal ovarian surface epithelial (HOSE) cells were established by scraping the surface of the ovary and growing recovered cells in a mixture of medium 199 and MCDB 105 medium supplemented with 10% fetal calf serum. The following seven normal HOSE cells were used: HOSE17, HOSE636, HOSE642, HOSE697, HOSE713, HOSE726 and HOSE730. Ovarian cell lines were established by recovery from ascites fluid or explanted from solid tumors. The following ten ovarian cancer cell lines were used: OVCA3, OVCA420, OVCA429, OVCA432, OVCA433, OVCA633, CAOV3, DOV13, ALST and SKOV3.

Serum specimens from women with ovarian cancer, other gynecologic cancers and benign gynecologic disorders requiring hysterectomy and from non-diseased normal women were obtained from discarded specimens, from discarded specimens that were archived during the period from 1983 through 1988 or from specimens collected under more recent protocols since 1996. The archived samples were collected from several studies assessing the performance of CA 125 in a variety of diagnostic circumstances, including gynecologically normal subjects as well as subjects having exploratory surgery for pelvic masses that proved to be ovarian, cervical or endometrial cancer for a benign disease such as a fibroid tumor. The archived specimens were stored at −70° C. However, thawing was known to have occurred once for some of the archived specimens. More recent specimens were obtained within the past five years and were stored at −70° C. without any incident of thawing. In both specimen banks, serum from case patients with ovarian cancer and serum from control patients were collected concurrently.

Microarray Probe and Hybridization

The MICROMAX™ Human cDNA Microarray System I(NEN Life Science Products, Inc. Boston, Mass.) was used in this study. Biotin-labeled cDNA was generated from 3 micrograms of total RNA that was pooled from HOSE17, HOSE636 and HOSE642 cells. Dinitrophenyl-labeled cDNA was generated from 3 micrograms of total RNA that was pooled from ovarian cancer cell lines OVCA420, OVCA433, and SKOV3. Before the cDNA reaction, 5 ng of Arabidopsis control RNA were added to each batch of the RNA samples for the normalization of hybridization signals. The biotin-labeled cDNA and the dinitrophenyl-labeled cDNA were mixed, dried and resuspended in 20 microliters of hybridization buffer 5× standard saline citrate (SSC), 0.1% sodium dodecyl sulfate (SDS) and salmon sperm DNA at 0.1 mg/ml (1×SSC=0.15 M NaCl, 0.15 M sodium citrate, pH 7). This mixture was added to the cDNA microarray and was covered with a coverslip. Hybridization was carried out overnight at 65° C. inside a hybridization cassette.

After hybridization, the microarray was washed with 30 ml of 0.5×SSC-0.01% SDS, with 30 ml of 0.06×SSC-0.01% SDS and then with 30 ml of 0.06×SSC alone. The hybridization signal from biotin-labeled cDNA was amplified with streptavidin-horseradish peroxidase and fluorescent dye, Cy5-tyramide. The hybridization signal from the dinitrophenyl-labeled cDNA was amplified with anti-dinitrophenyl-horseradish peroxidase and another fluorescent dye, Cy3-tyramide. After signal amplification and post-hybridization wash in TNT buffer (i.e., 0.1 M Tris-HCl (pH 7.5)-0.15 M NaCl-0.15% Tween20), the microarray was air-dried and signal amplification was detected with a laser scanner.

Laser detection of the Cy3 signal (derived from ovarian cancer cells) and the Cy5 signal (derived from HOSE cells) on the microarray was acquired with a confocal laser reader. Separate scans were taken for each fluor at a pixel size of 10 micrometers. cDNA derived from the added Arabidopsis RNA hybridized to 12 specific spots on the microarray, which were composed of DNA sequences obtained from four different Arabidopsis expressed sequence tags in triplicate. Cy3 and Cy5 signals from these 12 spots should theoretically be equal and were used to normalize the different efficiencies in labeling and detection with the two fluors. The fluorescence signal intensity and the ratio of the signals from Cy3 and Cy5 for each of the 2400 cDNAs were analyzed by the software Imagene 3.0 (Biodiscovery Inc., Los Angeles, Calif.).

Real-Time Quantitative Reverse Transcription-Polymerase Chain Reaction

Real-time reverse transcription-polymerase chain reaction (RT-PCR) was performed in duplicate by using primer sets specific for the overexpressed gene encoding the secretory protein prostasin (forward primer=5′-ACTTGAGCCACTCCTTCCTTCAG-3′ (SEQ ID NO:3); reverse primer=5′-CTGATGGTCCCAAAAAGCACAC-3′ (SEQ ID NO:4)) and a housekeeping gene, GADPH. RNA was first extracted from normal ovarian epithelial cell cultures (HOSE697, HOSE713, HOSE726, and HOSE730) and from 10 ovarian carcinoma cell lines (OVCA3, OVCA420, OVCA429, OVCA432, OVCA433, OVCA633, CAOV3, DOV13, SKOV3, and ALST). cDNA was generated from 1 microgram of total RNA using the TaqMan RT reagents containing 1× TaqMan reverse transcriptase buffer, 5.5 mM MgCl₂, all four deoxyribonucleoside triphosphates (each at 500 μM), 2.5 μM random hexamers, MultiScribe reverse transcriptase at 1.25 U/μl, and RNasin at 0.4 U/μl in 100 μl. The reaction was incubated at 25° C. for ten minutes at 48° C. for thirty minutes and finally at 95° C. for five minutes. A total of one microgram of cDNA was used in 20 μl PCR mixture containing 1×SYBR PCR buffer, 3 mM MgCl₂, all for deoxyribonucleoside triphosphates (each at 0.8 mM) and AmpliTaq Gold. The cDNAs were then amplified by denaturation for ten minutes at 95° C., followed by 40 PCR cycles of denaturation at 95° C. for 15 seconds and annealing-extension at 60° C. for one minute. The changes in fluorescence of the SYBR Green I dye in every cycle were monitored by ABI 5700 system software and the threshold cycle, which represents the PCR cycle at which an increase in reporter fluorescence above a baseline signal can first be detected for each reaction, was calculated. The relative amount of PCR products generated from each primer set was determined on the basis of the threshold cycle (C_{T}) value. GAPDH was used to normalize the quantity of RNA used. Its CT value was then subtracted from each target gene to obtain a ΔC_{T} value. The difference between the ΔC_{T} values of the samples for each gene target and the ΔC_{T} value of a calibrator which served as a physiologic reference was determined. For confirmation of the specificity of the PCR, PCR products were subjected to electrophoresis on a 1.2% agrose gel. A single PCR product with the expected size should be observed in samples that express the gene of interest.

Immunohistochemical Localization of Prostasin

Immunostaining with anti-prostasin antibody was performed on sections prepared from two normal ovaries, from two serous borderline ovarian tumors, and from two grade 1, two grade 2, and two grade 3 serous ovarian adenocarcinomas. This rapid polyclonal antibody, also used in the serum assay was prepared from prostasin purified from human seminal fluid as described previously (Yu, et al., J. Biol. Chem. 269:18843-18848 (1994)). Tissues were fixed in 4% paraformaldehyde and embedded in paraffin. Sections (5 μm) were cut, mounted on microscopic slides and incubated at 50° C. overnight. They were then transferred to Tris-buffered saline (TBS) and quenched in 0.2% H₂O₂ for 20 minutes. After quenching, the sections were washed in TBS for 20 minutes, incubated with normal horse serum for 20 minutes, and then incubated with anti-prostasin polyclonal antibody (diluted 1:400) at room temperature for one hour. The slides were then washed in TBS for 10 minutes, incubated with diluted biotinylated secondary horse anti-rabbit antibody solution for 30 minutes, washed again in TBS for 10 minutes, incubated with avidin-biotin complex reagent for 30 minutes and washed in TBS for 10 minutes. Stain development was performed for 5 minutes using a diaminobenzidine kit. Finally, the sections were washed in water for 10 minutes. They were then counterstained with hemanoxylin, dehydrated with an ascending series of alcohol solutions, cleared in xylene and mounted. The specificity of the staining was confirmed by using preimmunization rabbit serum and by preabsorbing the antibody with the purified peptide (60 mg/ml) or prostasin for 2 hours at 37° C. before applying the adsorbed antiserum to the sections.

Measurement of Prostasin and CA 125 in Sera

Sera were available from a total of 201 subjects (64 case patients with ovarian cancer and 137 control subjects, including 34 with other gynecologic cancers, 42 with benign gynecologic diseases, and 71 with no known gynecologic diseases). In all of the case patients and in the 68 control subjects who had surgery, preoperative specimens were available. Serum levels of immunoreactive human prostasin were determined by the enzyme-linked immunosorbent assay (ELICA) prepared with the previously described antibody to human prostasin. Microtiter plates (96 well) were coated with anti-prostasin immunoglobulin G (IgG) (1 μg/ml, 100 μl per well) overnight at 4° C. Purified prostasin standards or samples were added to individual wells in a total volume of 100 μl of phosphate-buffered saline containing 0.05% Tween 20 and 0.5% gelatin (dilution buffer) and incubated at 37° C. for 90 minutes. Biotin labeled anti-human prostasin IgG was added to each well at a concentration of 1 μg/ml in a total of 100 μl and incubated at 37° C. for 60 minutes. Peroxidase-avidin at a concentration of 1 μl/ml in a total volume of 100 μl was added and incubated at 37° C. for 30 minutes. The color reaction was performed by adding to each well 100 μl of freshly prepared substrate solution and 0.03% H₂O₂ in 0.1 M sodium citrate (pH 4.3) and incubating the mixture at room temperature for 30 minutes. The plates were read at 405 nm with a plate reader.

For 37 case patients with ovarian cancers and for 100 control subjects (about 70% of all subjects) a CA 125 level had been determined previously (from the same specimens) and was available for comparison. These measurements had been performed with the original CA 125 radioimmunoassay from Centocor and the assays were not repeated for this study.

Statistical Analysis

Univariate comparisons for quantitative variables between normal and cancer cell lines or between case and control sera were made using Student's t test. For analysis of serum levels, adjustment for potential confounding variables such as the subject's age, year of collection and whether the specimen had undergone freezing and thawing was carried out using general linear modeling. Logistic regression analysis was used to determine the statistical significance of both prostasin and CA 125 as a predictor of case status. Paired Student's t test was used to compare the change in postoperative prostasin levels from preoperative levels. Pearson correlation coefficients were calculated between CA 125 and prostasin. Because the distributions of prostasin and CA 125 were skewed positively, log-transformed values were used in statistical tests. Analyses with a P value of 0.05 or less were considered to be statistically significant. All statistical tests were two sided and all confidence intervals are 95%.

B. Results

Microarray analysis of pooled RNA isolated from three normal HOSE cell lines and from three ovarian cancer cell lines was performed. Thirty genes with Cy3/Cy5 signal ratios ranging from 5 to 444 were identified, suggesting that these genes were overexpressed in ovarian cancer cells compared with normal HOSE cells. Among them, both prostasin and osteopontin encode secretory proteins which may be potential serum markers. Another gene, creatine kinase B has been shown to produce a serum marker associated with renal carcinoma and lung cancer. Prostasin was selected for further study because this gene had an available antibody assay.

To evaluate the differential expression of prostasin in individual normal and malignant ovarian epithelial cell lines derived from normal and neoplastic ovaries, we performed quantitative PCR analysis on four normal HOSE cultures and on ten ovarian cancer cell lines. The relative prostasin gene expression ranged from 120.3-fold to 410.1-fold greater for seven of the ten ovarian cancer cell lines compared with that for HOSE 697 cells but was only marginally greater for three other ovarian cell lines. Overall, there was a highly statistically significant difference between expression for the four normal cell lines compared to the ten ovarian cancer cell lines P<0.001.

For further validation of the expression of prostasin in actual tumor tissue, sections from two normal ovaries, from two serous borderline ovarian tumors and from two grade 1, two grade 2 and two grade 3 serous ovarian cystadenocarcinomas were immunostained with an anti-prostasin polyclonal antibody. Stronger cytoplasmic staining was detected in cancer cells than in normal HOSE cells, suggesting that prostasin is overexpressed by the ovarian cancer cells. Prostasin was, however, also detected in normal ovarian tissue by immunostaining. We next examined prostasin levels detected by ELISA in sera from case patients and control subjects. The mean prostasin level for all of the case patients was 13.7 μg/ml compared with 7.5 μg/ml in all of the control subjects. Based on log-transformed values, this difference was statistically significant (P<0.001) and persisted after adjustment for the subject's age, year of collection, and quality of specimen (possible freeze-thaw damage). Among case patients, there was considerable variability by stage; however, notably women with stage II disease had the highest levels of prostasin, suggesting that prostasin may be of use for early-stage detection. It also appeared that women with mucinous-type ovarian tumors had lower levels of prostasin than women with ovarian tumors of other epithelial types. Among control subjects, there was a statistically significant tendency for the archived specimens to have lower prostasin levels than the current specimens (P<0.001), but there was no evidence for an effect of age or diagnostic category (i.e., normal tissue, benign gynecologic disease, or other gynecologic cancer). In addition, 60.5% of the archived case specimens and 66.2% of the control specimens had been in the freezer in which freezing and thawing had occurred. There was no evidence of a tendency for these samples to have lower prostasin levels.

In sixteen women with nonmucinous epithelial ovarian cancers, preoperative and postoperative specimens were available for comparison. For fourteen of these women, a decreased prostasin level was observed after surgery, and, in the entire group of sixteen, postoperative P levels were statistically lower when compared with preoperative levels using a pair T test on the log-transformed values (P=0.004).

A bivariate plot of prostasin versus CA 125 performed for the 37 case patients with nonmucinous ovarian cancers and for the 100 control subjects who had both measurements available failed to show a statistically significant correlation. This lack of correlation suggests that the two markers may provide complementary information. The combined markers had a sensitivity of 34/37 (92%) and a specificity of 94/100 (94%). In contrast, the sensitivity of CA 125 alone at the same specificity was 24/37 (64.9%) and the sensitivity of prostasin alone at the same specificity was 19/37 (51.4%).

C. Discussion

The present study demonstrates prostasin's potential as a biomarker through real-time PCR in cancer and normal epithelial cell lines and by differential staining in cancer tissue compared with normal tissue. Higher levels of serum prostasin were found to be present in case patients with ovarian cancer when compared to control subjects and a declining level of prostasin was observed after surgery for ovarian cancer. Results also suggest that assays with prostasin may be combined with those for other markers such as CA 125 to improve the reliability of procedures for the detection of ovarian cancer.

### Example 4

### Identification of Eosinophil-Derived Neurotoxin (EDN) and Osteopontin as Urine Biomarkers for Ovarian Cancer

Surface enhanced laser desoprtion/ionization-mass spectrometry (SELDI-MS) and two dimensional electrophoresis were used for initial urine biomarker screening followed by LC-MS/MS (liquid chromatography-tandem mass spectrometry) to identify protein sequences. Four different types of surface specific protein chip arrays were used for screening protein biomarkers with molecular weights of less than 50 kDa. These included IMAC3 (immobilized metal affinity capture), H4 (hydrophobic surface), SAX2 (strong anion exchanger), and WCX2 (weak cation exchanger). The preliminary study used a total of 145 urine specimens collected pre-operatively. These specimens included 48 from patients with age-matched benign tumors, 42 from ovarian cancer cases, and 55 from healthy women. Using the WCX2 chip, a candidate protein was identified with a molecular weight of 17.4 kDa. This was consistently expressed to a higher degree in urine from cancer patients (peak intensity mean=13.8) and in urine from patients with benign tumors (7.72) than in urine from normal women (0.55, p<0.05). Using the mean plus standard deviation as a cutoff, this protein can be used to distinguish ovarian cancer cases from normal cases with 96% specificity and 86% sensitivity. It was less sensitive for benign tumors (56% sensitivity). The protein peak was further purified by liquid chromatography and characterized by LC-MS/MS. The C-terminal fragment sequence of the protein was identical to eosinophil-derived neurotoxin (EDN). The identity of the protein was also validated by Western blot. A total of 219 urine samples were used for further ELISA validation. This included 88 samples from normal individuals, 56 samples from patients with benign tumors, and 75 samples from patients with ovarian cancer (31 of stage I/II and 44 at stage III/IV). The final concentration of urine EDN was normalized as ng per mg of total protein. Using the standardized ELISA assay, this urine protein marker resulted in 83% specificity, 71% sensitivity for early stage disease, and 75% sensitivity for late stage disease.

Using a two dimensional gel electrophoresis system, a cluster of protein spots were identified as being overexpressed in urine samples collected from different subtypes of ovarian cancers. These spots were subjected to LC-MS/MS sequence analysis and identified as osteopontin polypeptides. Further validation of the identity of these proteins was obtained by Western blots using osteopontin antibodies. A commercially available ELISA assay of urine samples from the age-matched 88 samples from normal controls, 29 samples from patients with benign ovarian tumors, and 56 samples from ovarian cancer patients was performed and showed 92% specificity and 62% sensitivity. The results suggest that urinary EDN and osteopontin with their modifications, i.e., glycosylation, can serve as urinary biomarkers for ovarian cancer detection. It is believed that a more sensitive and specific separation of early stages of ovarian cancer may be obtained by combining this urine marker with other serum biomarkers such as CA 125.

All references cited herein are fully incorporated by reference. Having now fully described the invention, it will be understood by those of skill in the art that the invention may be performed within a wide and equivalent range of conditions, parameters and the like, without affecting the spirit or scope of the invention or any embodiment thereof.

1. A method of diagnostically evaluating a woman for the presence of ovarian cancer, comprising:
(a) obtaining a urine sample from said woman;
(b) assaying said urine sample for the concentration of osteopontin present;
(c) comparing the results obtained from the assay of step (b) with results obtained from the assay of one or more control samples; and
(d) concluding that said woman is at increased risk of having ovarian cancer if the concentration of osteopontin in said urine sample is higher than the concentration in said control sample or samples.

2. The method of claim 1, wherein the concentration of osteopontin present in said urine sample is determined by an immunoassay.

3. The method of claim 1, wherein the concentration of osteopontin in said urine sample is determined by surface enhanced laser desorption/ionization-mass spectrometry.

4. The method of claim 1, wherein the concentration of osteopontin in said urine sample is determined by chromatography.

5. The method of claim 1, wherein the concentration of osteopontin in said urine sample is determined by using electrophoresis.

6. The method of claim 1, wherein the concentration of osteopontin in said urine sample is determined using an ELISA.

7. The method of claim 1, wherein said one or more control samples are urine samples obtained from women believed not to have a malignant disease.

8. The method of claim 1, wherein the results from said urine sample is compared to the results obtained from a group of samples taken from the general population.

9. The method of any one of claims 1-8, wherein it is concluded that a woman is at increased risk of having ovarian cancer if the osteopontin concentration in said urine sample is higher than the amount in said control sample or samples by at least one standard deviation.

10. The method of any one of claims 1-8, further comprising performing at least one additional assay for a diagnostic marker of cancer.

11. The method of claim 10, wherein said diagnostic marker of cancer is CA 125.
